(19) United States
(12) Patent Application Publication
(10) Pub. No.: US 2024/0350712 A1
(43) Pub. Date: Oct. 24, 2024

(54) ALGINATE HYDROGEL MICROSTRANDS FOR STROMAL CELL ENCAPSULATION, MAINTENANCE AND IMPLANTATION

(71) Applicant: The Research Foundation for the State University of New York, Albany, NY (US)

(72) Inventors: Yubing Xie, Albany, NY (US); Sujith Kollampally, Albany, NY (US); Matthew Jorgensen, Albany, NY (US)

(21) Appl. No.: 18/633,107

(22) Filed: Apr. 11, 2024

Related U.S. Application Data
(63) Continuation-in-part of application No. 17/153,867, filed on Jan. 20, 2021.
(60) Provisional application No. 62/963,517, filed on Jan. 20, 2020.
(60) Provisional application No. 63/458,539, filed on Apr. 11, 2023.

(51) Int. Cl. A61L 27/52; A61M 5/24

(52) U.S. Cl. CPC A61L 27/52; A61M 5/2448

(57) ABSTRACT

A device and process for fabricating alginate hydrogel microstrands, and product produced thereby, that are advantageously used for stromal cell encapsulation and maintenance. The device has first reservoir including a cell-alginate solution therein that selectively intakes and expels the cell-alginate solution therefrom, and a second reservoir in fluid connection with the first reservoir, the second reservoir containing a cross-linker solution and selectively intakes the cell-alginate solution from the first reservoir and expels the combined cell-alginate solution and cross-linker solution therefrom. The second reservoir selectively expels the combined cell-alginate solution and cross-linker solution as cell-laden hydrogel microstrands. The reservoirs of the device can be interconnected syringes.

### CROSS-REFERENCE TO RELATED APPLICATIONS

This application is a continuation-in-part of U.S. patent application Ser. No. 17/153,867, filed on Jan. 20, 2021, which claims the benefit of U.S. Provisional Patent Application No. 62/963,517, filed on Jan. 20, 2020, and this application also claims the benefit of U.S. Provisional Patent Application No. 63/458,539, filed Apr. 11, 2023, the entireties of which are hereby incorporated herein by this reference.

### BACKGROUND OF THE INVENTION

### 1. Field of the Invention

The present invention generally relates to the engineering of biological structures. More particularly, the present invention relates to the creation of microstrands from alginate hydrogel for the encapsulation and maintenance of mesenchymal stromal cells.

### 2. Description of the Related Art

Mesenchymal stromal cells (MSCs) are multipotent stem cells that can be isolated from various tissues, including bone marrow, adipose tissue, and umbilical cord and are of interest for their re-generative responses in vivo. Apart from their multilineage differentiation capacity, MSCs have been shown to possess immunomodulatory and anti-inflammatory properties, capability of secreting bioactive molecules for tissue repair, and the ability to modulate fibrotic responses. These diverse characteristics make MSC-based cell delivery of interest for therapeutics for bone, cartilage, ligament, skin, heart, and other organs.

Since MSCs can home to and target the inflamed tissue, their survival, retention and engraftment play a crucial role in their utilization as therapeutic agents, including localized and targeted therapies. However, transplanted MSCs face many shortcomings, one of which is low MSC survival, retention and homing efficiency post-delivery due to undesired biodistribution in host, for example, being entrapped by the lung. This challenge highlights the need for delivery vehicles that can improve the survival and local retention of MSCs, which may enhance their regenerative, and anti-inflammatory/fibrotic properties.

Hydrogels have shown feasibility for delivering MSCs in vivo and demonstrated improved MSC survival and retention. And in particular, alginate hydrogels have been explored in different structures for MSC delivery, such as discs or sheet-like gels, microbeads, microfibers, and 3D printed geometries. These studies have shown some benefits of enhanced cell retention and implanted cell survival. Among these alginate hydrogels, microtubular structures with diameters that range from 20 to 800 μm (e.g., microtubes, microfibers, microribbons, microstrands) have been used for long term cultures, which maintain good cell viability, and show great potential to support cell proliferation and function in vitro and in vivo. The high porosity of tubular hydrogels offers a large surface area and space for cellular organization, proliferation, and expansion in vitro. However, it is challenging to use existing methods to generate small volumes of microstrands with high cell densities for cell implantation for useful testing in animal models.

An optimal delivery vehicle for generating hydrogels to deliver a significant volume of MSCs should maximize the regenerative efficacy while maintaining the viability, phenotype, and functionality of the MSCs. It is thus to the creation of such hydrogels and methods of their use as a delivery vehicle of MSCs that the present invention is primarily directed.

### BRIEF SUMMARY OF THE INVENTION

Briefly described, the present invention includes a device and process for fabricating alginate hydrogel microstrands, and a product produced thereby, that are advantageously used for stromal cell encapsulation and maintenance. Here, the device and process produce three-dimensional (3D), cell-laden alginate hydrogel microstrands with long, thin fiber-like structures, featuring diameters that range from 100 to 300 μm. The thin fiber structure facilitates efficient radial diffusion of nutrients and oxygen across the microstrands for stromal cell growth and differentiation. The long fiber structure facilitates handling and retrieving of the cell-laden microstrands, enabling improved cell delivery of MSCs for tissue regeneration studies.

Prior approaches to fabricating cell-laden hydrogel microstrands, microfibers and core-shell microtubes for the use in clinical studies and settings include microfluidics wet spinning, extrusion and 3D printing. However, it is very challenging to specifically use these methods to generate small volumes of cell-laden hydrogel microstrands with high cell densities for cell implantation in small animal models.

The present invention, in one embodiment, is a Syringe-in-Syringe (SiS) device to fabricate alginate hydrogel microstrands in small volumes suitable for cell implantation in mouse models. The device assembly and process of use fabricates microstrands containing stromal cells using alginate hydrogels to support MSC growth and viability and maintain their anti-fibrotic properties.

In one embodiment, NIH 3T3 cells are used for cell encapsulation and initial cell seeding density and growth in microstrands. Due to their MSC-like properties and their ability to support early differentiation of salivary gland epithelium, the advantageous survival and phenotypic maintenance for primary embryonic 16 (E16) salivary mesenchyme cells in the microstrands can be demonstrated.

In one embodiment, the invention includes a device for fabricating alginate hydrogel microstrands that has a first reservoir, which will hold a cell-alginate solution therein selectively intake and expel the cell-alginate solution therefrom, and a second reservoir in fluid connection with the first reservoir, the second reservoir having a cross-linker solution and is configured to selectively intake the cell-alginate solution from the first reservoir and expel the combined cell-alginate solution and cross-linker solution therefrom. The second reservoir selectively expels the combined cell-alginate solution and cross-linker solution as cell-laden hydrogel microstrands. The expulsion of the solution can be adjacent to one or more mammalian cells in vivo or ex vivo in a laboratory setting, such as in a cell culture.

The device can be embodied with the second reservoir selectively detaching at a nozzle from the first reservoir, and the nozzle configured to expel the cell-laden hydrogel microstrands adjacent mammalian cells. Further, the cell-alginate solution and cross-linker solution can be combined at a predetermined duration, such as 5 to 50 seconds, to form cell-laden alginate hydrogel microstrands with long, thin fiber-like structures. Further, the cell-alginate solution can include NIH 3T3 cell, and the cross-linker solution can include CaCl₂).

In one embodiment, the first reservoir and second reservoir are each a syringe having manually operated plungers therein, and each syringe has a nozzle. The first nozzle of the first syringe and second nozzle of the second syringe are selectively attached to thereby create a fluid coupling between the first chamber and second chamber. There can also be a filter having micropatterned pores connecting the first reservoir and second reservoir.

In one embodiment, the invention includes a process for fabricating alginate hydrogel microstrands, starting by placing a cell-alginate solution a first reservoir that is configured to selectively intake and expel the cell-alginate solution therefrom, then placing a cross-linker solution in a second reservoir in fluid connection with the first reservoir, the second reservoir configured to selectively intake the cell-alginate solution from the first reservoir and expel the combined cell-alginate solution and cross-linker solution therefrom. The process then continues with the intaking the cell-alginate solution from the first reservoir into the second reservoir, allowing the cell-alginate solution and the cross-linker solution to mix for a predetermined duration, and after the predetermined duration, expelling the combined cell-alginate solution and cross-linker solution as cell-laden hydrogel microstrands from the second reservoir. When the device is so configured with the second reservoir selectively detaching at a nozzle from the first reservoir, the method further includes detaching the nozzle from the first reservoir and expelling the cell-laden hydrogel microstrands adjacent to one or more mammalian cells.

The present invention is therefore advantageous as it provides a device, process, and product of alginate hydrogel microstrands that are advantageously used for stromal cell encapsulation and maintenance in clinical laboratory settings, specifically with mammalian models. The invention is industrially applicable in that it provides a laboratory device and process of use in laboratory studies as well as in situ on mammalian cells. Other advantages, benefits, and application would be apparent to one of skill in the art from reviewing the disclosure of the present application.

### BRIEF DESCRIPTION OF THE DRAWINGS

FIG. 1 is a diagram of one embodiment of the device for preparing alginate hydrogel microstrands.

FIG. 2 is a series of optical images of alginate hydrogel microstrands fabricated by the device in FIG. 1.

FIG. 3 is a graph illustrating the efficiency of cell encapsulation and recovery from alginate hydrogel microstrands containing NIH 3T3 fibroblasts at different initial cell seeding densities.

FIG. 4A is a graph illustrating viability of cells cultured in alginate hydrogel microstrands with NIH 3T3 fibroblasts encapsulated in microstrands at various initial cell seeding densities.

FIG. 4B is a graph illustrating viability of cells cultured in alginate hydrogel microstrands with primary E16 mesenchyme cells encapsulated in microstrands at various cell seeding densities.

FIG. 5A is a graph illustrating cell growth of NIH 3T3 fibroblasts in alginate hydrogel microstrands at different initial cell seeding densities through viable cell number.

FIG. 5B is a graph illustrating cell growth of NIH 3T3 fibroblasts in alginate hydrogel microstrands at different initial cell seeding through cell expansion in fold.

FIG. 6 is a graph illustrating cell growth of primary E16 mesenchyme cells in alginate hydrogel microstrands.

FIG. 7 is a series of confocal images of primary E16 mesenchyme cells cultured in alginate hydrogel microstrands.

### DETAILED DESCRIPTION OF THE INVENTION

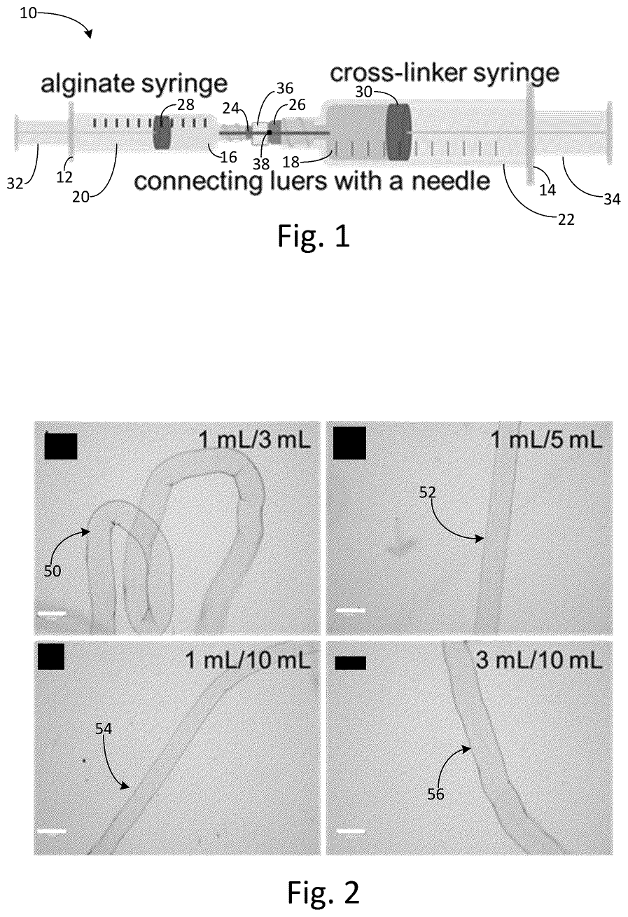

With reference to the figures in which like numerals represent like elements throughout the several views, the present invention provides a device 10 (FIG. 1) and process for fabricating alginate hydrogel microstrands, and a product produced thereby, that are advantageously used for stromal cell encapsulation and maintenance. Alginate hydrogels can support the delivery of MSCs both in ex vivo and in vivo settings. Alginate is a naturally occurring anionic polysaccharide heteropolymer derived from brown seaweed, consisting of alternating blocks of (1,4)-linked β-D-mannuronate (M) and its C5-epimer, α-L-guluronate (G). The composition of these blocks influences the physical and chemical properties of alginate. Alginate's ability to form hydrogels in the presence of divalent cations, such as Ca²⁺ and Ba²⁺, is attributed to the carboxylate groups present in its structure. The crosslinking network of alginate hydrogels enables them to simulate ECM-like characteristics such as high-water content, porosity, permeability, and viscoelasticity. The advantages of alginate hydrogels, including ease of chemical modification (addition of bioactive cues and therapeutic agents), support for cell viability (biocompatibility, mild gelation and soft-tissue-like mechanical properties), and ease of transplantation (biodegradability and malleability, have made it a popular biomaterial for drug delivery systems, tissue engineering scaffolds, and encapsulation of living cells.

For the preparation of one embodiment of the alginate hydrogel solution, sodium alginate (FUJIFILM Wako Pure Chemicals Co., Osaka, Japan) was dissolved in 0.9% sodium chloride solution (NaCl, Sigma-Aldrich, St Louis, MO, USA) at a concentration of 1.5% w/v or 3% w/v and then autoclaved at 121° C. for 15 minutes. The sterilized solutions were stored at 4° C. for future use. 100 mM CaCl₂) (Sigma-Aldrich) solution based on previous optimization studies was prepared and autoclaved at 121° C. for 15 minutes and stored at room temperature for future use.

The viscosity of alginate solution was measured using a BS/U capillary Ostwald viscometer (DC Scientific, Glen Burnie, MD, USA). Briefly, 1.5% w/v alginate solution was injected into the U-shaped Ostwald viscometer and the time required to pass through the bulb due to the capillary hydrostatic pressure was measured. The kinematic viscosity (v) (mm²/s) was determined by multiplying the constant of the viscometer (C=0.0272) and the transit time (t) as shown as:

v=C×t

With reference to FIG. 1, in one embodiment, the device 10 for fabricating alginate hydrogel microstrands that has a first reservoir (syringe 12), which will hold a cell-alginate solution 16 therein, in a first chamber 20, and the first reservoir selectively intake and expel the cell-alginate solution 16 therefrom. A second reservoir (syringe 14) is in fluid connection with the first reservoir (syringe 12), attached here as a coupling 24 to the first nozzle 24 of the first reservoir, and the second nozzle 26 of the second reservoir. The second reservoir (syringe 14) has a cross-linker solution 18 in its chamber 22 and is configured to selectively intake the cell-alginate solution 16 from the first reservoir (syringe 12) and expel the combined cell-alginate solution 16 and cross-linker solution 18 therefrom. The second reservoir (syringe 14) selectively expels the combined cell-alginate solution 16 and cross-linker solution 18 as cell-laden hydrogel microstrands (see FIG. 2). The expulsion of the combined solution can be adjacent to one or more mammalian cells in in vivo or ex vivo in a laboratory setting, such as in a cell culture. The cross-linker solution 18 can include one of Ca²⁺·CaCl₂, Ba²⁺·Sr²⁺.

The device 10 can be embodied as shown in FIG. 1, with the second reservoir (syringe 14) selectively detaching at a second nozzle 26 from the first reservoir, and the second nozzle 26 can be configured to expel the cell-laden hydrogel microstrands adjacent mammalian cells. Further, as further discussed herein, the cell-alginate solution 16 and cross-linker solution 18 can be combined at a predetermined duration, such as 5 to 50 seconds, to form cell-laden alginate hydrogel microstrands with long, thin fiber-like structures.

Further as shown in the embodiment of FIG. 1, the first reservoir is a syringe 12 and second reservoir is a syringe 14, with each a syringe having manually operated plungers therein (first plunger 28 with first piston 32; second plunger 30 with second piston 34). Here, each syringe includes a nozzle with the first nozzle 24 of the first syringe 12 and second nozzle 26 of the second syringe 14 are selectively attached to thereby create a fluid coupling between the first chamber 20 and second chamber 22.

Also, in the embodiment of FIG. 1, there can be a filter 38 that can filter the combined alginate solution 16/cross-linker solution 18 as it is either expelled from the second syringe 14, or filter the alginate solution 16 at intake. The filter 38 can have micropatterned pores only allowing a certain size of cell therethough.

In FIG. 1, the syringe-in-syringe device contains two syringes (first syringe 12 and second syringe 14) (BD, Franklin Lakes, NJ) of either the same volume or different volumes. A needle (blunt end) (Hamilton Company, Reno, NV, USA) with plastic or steel luer locks was used to bridge the two syringes with the aid of an adapter 36 (Cole Parmer, WA, USA).

In one embodiment of use to fabricate alginate hydrogel microstrands suitable for cell implantation studies, an alginate solution of 1.5% w/v was loaded into the first syringe 12 (alginate syringe), while the other second syringe 14 (cross-linker syringe) was loaded with 100 mM CaCl₂). The second plunger 30 on the cross-linker second syringe 14 was then pulled at piston 34, creating a negative pressure along the needle bridge to draw the alginate solution 16 from the alginate first syringe 12 into the cross-linker second syringe 14. As the alginate solution 16 encountered the calcium ions of the cross-linker solution 18 in the cross-linker second syringe 14, immediate crosslinking occurred to form alginate hydrogel microstrands. Different syringe volume/capacity combinations of SiS devices were constructed, including 1 mL alginate syringe/1 mL cross-linker syringe, 1 mL/3 mL, 1 mL/5 mL, 1 mL/10 mL, 3 mL/3 mL, 3 mL/5 mL, 3 mL/10 mL, 5 mL/5 mL, and 5 mL/10 mL, connected with a blunt end needle (30G). The volume of 1.5% w/v alginate solution in the alginate syringe was kept constant (250 μL) and the volume of 100 mM CaCl₂) solution was approximately 70% of the cross-linker syringe capacity (e.g., 0.7 mL CaCl₂) for 1 mL cross-linker syringe, 2.3 mL CaCl₂) for 3 ml syringe, etc.). The feasible and optimal conditions to fabricate 250 μL alginate hydrogel microstrands with diameter around 200 μm were investigated.

Murine NIH 3T3 fibroblasts were maintained in high glucose Dulbecco's Modified Eagle's Medium (DMEM) (Sigma-Aldrich), supplemented with 10% fetal bovine serum (FBS) (Sigma-Aldrich) and 1% penicillin-streptomycin (pen/strep, 10,000 units penicillin/10 mg streptomycin from Sigma-Aldrich), in a 37° C., 5% CO₂ humidified incubator. The medium was changed one day before subculture, and subculture was performed every two to three days.

Murine primary E16 mesenchyme cells were isolated from salivary glands of embryonic day 16 (E16) timed-pregnant CD-1 female mice from Charles River Laboratories (Wilmington, MA, USA). The care and handling of mice was carried out in accordance with the National Institutes of Health Guide for the Care and Use of Laboratory Animals and with protocols approved by the Institutional Animal Care and Use Committee (IACUC) of the University at Albany, State University of New York. Primary E16 mesenchyme cells were cultured in DMEM/F12 (Sigma-Aldrich), supplemented with 10% fetal bovine serum (FBS) and 1% pen/strep for 4 days in a 37° C., 5% CO₂ humidified incubator. The medium was changed on day 1 and day 3.

Murine NIH 3T3 fibroblasts were used as a stromal cell model to generate 250 μL of alginate hydrogel microstrands for cell encapsulation, future implantation studies, and to determine encapsulation, recovery efficiency, cell viability and growth in these microstrands. Briefly, 125 μL of 3% w/v sterile alginate solution was mixed well with 125 μL of cell culture medium (DMEM+10% FBS+1% pen/strep) to yield a final alginate concentration of 1.5%. NIH 3T3 fibroblasts were trypsinized with 0.05% trypsin/EDTA (Sigma Aldrich) and neutralized with FBS in cell culture medium. 0.25, 0.5, 1, 2, or 5×10⁶ cells were aliquoted into 15 mL sterile centrifuge tubes, respectively, and centrifuged at 1200 rpm for 5 min. After carefully removing the supernatant, 250 μL of 1.5% alginate-medium solution was added to the cell pellets and gently pipetted up and down to ensure uniform mixing of cells with the alginate-medium solution, resulting in initial cell seeding densities of 1, 2, 4, 8, and 20×10⁶ cells/mL alginate solution, respectively.

The cell-alginate solution was then loaded into a sterile 1-mL BD syringe, i.e., the alginate first syringe. 100 mM CaCl₂) solution was loaded into a 5-mL BD syringe, i.e., the cross-linker syringe 12. Both syringes 12, 14 were then joined using an adapter 36 with a blunt ended needle (30G) that bridges both solutions 16,18 in each syringe. The second plunger 30 on the cross-linker syringe was then pulled by piston 34, creating a negative pressure to draw the cell-alginate solution 16 from the alginate first syringe 12 into the CaCl₂) cross-linker solution 18 in the cross-linker second syringe 14 to form cell-laden alginate hydrogel microstrands. The cell-laden hydrogel microstrands were maintained in the CaCl₂) solution for 10-20 seconds (but could be in a range of 5 to 50 seconds) and then transferred to a 70 μm Falcon cell strainer (Thermo Fisher Scientific, Pittsburgh, PA, USA), followed by immersion in 0.9% NaCl solution to remove excess CaCl₂). The cell strainer containing cell-laden hydrogel microstrands was then transferred to a six-well plate with 6 mL of media for NIH 3T3 fibroblasts (DMEM+10% FBS+1% pen/strep), supplemented with 25 mM CaCl₂) to maintain the hydrogel integrity during cell culture. Six ml of medium was added to each well to ensure that the microstrands in the cell strainer were fully immersed in the medium. The microstrands were evenly distributed in the cell strainer fitted in the six-well plates, allowing proper diffusion of oxygen and nutrients into the microstrands. The medium was changed daily, leaving 0.5 mL conditioned medium and adding 5.5 mL fresh medium to allow cell growth for 4 days.

Primary E16 salivary mesenchyme cells were also encapsulated in alginate hydrogel microstrands in the same manner as the NIH 3T3 cells except that E16 mesenchyme cell pellets contained 2.5×10⁵, 5×10⁵ and 1×10⁶ cells, respectively. E16 mesenchyme cell-laden hydrogel microstrands at initial cell seeding densities of 1, 2, and 4×10⁶ cells/mL alginate solution were prepared as described above, transferred in a cell strainer fitted in a six-well plate, and cultured in 6 mL of media (DMEM/F12+10% FBS+1% pen/strep) supplemented with 25 mM CaCl₂) for 3 days to evaluate cell viability and growth and for 4 days to evaluate mesenchymal marker expression.

As shown in FIG. 2, Optical images of microstrands were taken using an EVOS M7000 Imaging System (Themo Fisher Scientific). The diameter of the hydrogel microstrand was measured using EVOS M700 image analysis software and calculated as the mean±standard deviation. Optical images of cell growth in alginate hydrogel microstrands were also acquired using an EVOS M7000 Imaging System on days 0, 1, 3 and 4. FIG. 2 shows optical images of alginate hydrogel microstrands fabricated by SiS device with alginate first syringe 12 volume/capacity to cross-linker syringe volume/capacity of 1 mL/3 mL. Image (A) shows microstrands 50 at 1 mL/3 mL; Image (B) shows microstrands 52 at 1 mL/5 mL; Image (C) shows microstrands at 1 mL/10 mL; Image (D) shows microstrands at 3 mL/10 mL. The scale bar in FIG. 2 is 300 μm.

A trypan blue exclusion assay was used to determine cell viability before cell encapsulation in alginate hydrogel microstrands and after cell culture in the microstrands. At the conclusion of each cell culture period, the cell-laden microstrands, which were placed on a cell strainer in a six-well plate, were first removed from their cell media and then rinsed with 0.9% NaCl solution. Subsequently, the microstrands were transferred to a new six-well plate. To dissolve the microstrands, 1-2 mL of 55 mM sodium citrate solution was added for 5-10 min, followed by adding 1-2 mL of fresh cell culture media. For the trypan blue staining, equal volumes of the cell suspension and 0.40% Trypan Blue Dye (BIO-RAD, Hercules, CA, USA) were mixed by gently pipetting up and down 10 times in a sterile microcentrifuge tube. 10 μL of this mixture was loaded to each side of a Brightline hemocytometer (Sigma-Aldrich), allowing the counting of unstained viable cells and blue-stained dead cells in the four outer grids using a manual cell counter.

Cell viability in microstrands was further confirmed by Invitrogen LIVE/DEAD Viability/Cytotoxicity kit (Thermo Fisher Scientific) followed by fluorescence imaging using the EVOS M7000 Imaging System. On day 4, cell-laden microstrands in the cell strainer were incubated with 2 M calcein AM and 4 μM ethidium homodimer-1 (EthD-1) in 6 mL of cell culture media at room temperature for 30 min. Microstrands were then transferred to a Platinum Line cover glass slide (25×75 mm) (Waldemar Knittel Glasbearbeitungs, Braunschweig, Germany) and observed with a EVOS M7000 Imaging System with an EVOS LIGHT CUBE, GFP 2.0 (excitation at 494 nm/emission at 540 nm) to visualize live cells that convert calcein AM to green fluorescent calcein and with an EVOS Light Cube, Texas Red 2.0 (excitation at 595 nm/emission at 613 nm) to visualize red fluorescent EthD-1-stained dead cells.

To quantify NIH 3T3 fibroblasts and primary E16 mesenchyme cells in alginate hydrogel microstrands from different initial cell seeding densities over a period of time, an alamarBlue assay (Thermo Fisher Scientific) was performed. To optimize the alamarBlue assay for cells grown in alginate hydrogel microstrands, we first encapsulated 150,000, 300,000, 600,000, and 1,000,000 cells in 250 μL microstrands, respectively, and added 120 UL or 600 μL alamarBlue solution to 6 mL media. In both cases the standard curve showed high linearity R₂>0.99. To determine cell growth in microstrands, we chose to add 120 μL alamarBlue solution to 6 mL media (2%) for the assay to save the reagent, and in particular, to avoid potential cytotoxicity effect of the alamarBlue dye [73-75].

For NIH 3T3 fibroblasts, cells were encapsulated in 250 μL alginate hydrogel microstrands at initial cell seeding densities of 1, 2, 4, 8, and 20×10⁶ cells/mL alginate solution, respectively, and cultured in a humidified incubator at 37° C., 5% CO₂ for 3 hours (Day 0) and 1-4 days (Day 1, Day 2, Day 3, and Day 4). For primary E16 mesenchyme cells, cells were encapsulated in 250 μL alginate hydrogel microstrands at initial cell seeding densities of 1, 2, and 4×10⁶ cells/mL alginate solution, respectively, and cultured in a humidified incubator at 37° C., 5% CO₂ for 3 hours (Day 0) and 1-3 days (Day 1 and Day 3). To prepare the standard curve for each time point, 0.25, 0.5, 1, 2, and 5×10⁶ viable NIH 3T3 fibroblasts were encapsulated in 250 μL alginate hydrogel microstrands and incubated in media at 37° C., 5% CO₂ for 3 hours. The predetermined incubation duration can be in a range of 1 to 8 hours.

To initiate alamarBlue assay, 6 mL fresh medium was added to the microstrands present on the cell strainer fitted in each well of the six-well plate. Then, 120 μL of alamarBlue reagent was added to each well. After incubation for six hours, 100 μL of medium in triplicate was taken from the standard curve plates and sample plates, respectively, and loaded into the same 96-well plate. The plate was then read for fluorescence intensity in relative fluorescence units (RFU) at excitation/emission 545 nm/590 nm with negative controls (medium only and microstrands only) using Infinite® 200 PRO microplate reader (Tecan, Mannedorf, Switzerland). The standard curve of fluorescence intensity (RFU) vs. viable cell number was plotted. Subsequently a linear regression equation was obtained from each standard curve. The cell number in microstrands was calculated based on the standard curve. We then validated our alamarBlue assay for measuring cell number in high density alginate hydrogel microstrands by plotting the measured cell number using alamarBlue assay vs. cell number from trypan blue cell counting, which showed good correlation with slope=0.9926 and R²=0.9903.

To evaluate the protein expression by cells in alginate hydrogel microstrands, immunocytochemistry was performed. Cell-laden hydrogel microstrands in cell strainers fitted in a six-well plate were rinsed with 0.9% NaCl and then fixed in 4% paraformaldehyde (Sigma-Aldrich) in 0.9% NaCl for 30 min on ice. Samples were then rinsed with 0.1% Tween 20 (Thermo Fisher Scientific) in 0.9% NaCl three times, rocking at 40 rpm for 5 min each time. The cell-laden microstrands were then permeabilized in 0.1% Triton X-100 (Sigma-Aldrich) in 0.9% NaCl rocking at 40 rpm for 15 min at room temperature. Three more wash steps were performed with 0.1% Tween 20 in 0.9% NaCl solution, followed by blocking with 20% MilliporeSigma donkey serum (Sigma-Aldrich, Burlington, MA, USA) in 0.9% NaCl for 1-2 hours at room temperature. The primary antibody was diluted in 3% bovine serum albumin (BSA) (Thermo Fisher Scientific) in 0.9% NaCl and incubated with cell-laden microstrands overnight at 4° C. on a rocker. Antibodies used were anti-vimentin (1:400, V2258, Sigma-Aldrich), anti-PDGFRa (1:100, AF1062, R&D systems) and anti-α-SMA (1:400, A5228, Sigma-Aldrich). After washing with 0.1% Tween 20 in 0.9% NaCl for four times with 10 minutes rocking per wash, the respective secondary antibodies along with 4′,6-diamidino-2-phenylindole (DAPI) (1:400, Sigma-Aldrich) was added to each sample and incubated for 2 hours on a rocker at room temperature. Alexa Fluor® 647 donkey anti-mouse IgG (1:250, 715-606-150, Jackson ImmunoResearch Laboratories, West Grove, PA, USA for vimentin and α-SMA) or Alexa Fluor® 488 donkey anti-goat IgG (1-250, 705-545-147, Jackson ImmunoResearch Laboratories for PDGFRa), respectively. The microstrands were washed with 0.1% Tween 20 in 0.9% NaCl three more times before mounting them on slides with EMS Fluoro-Gel mounting media (1:100 PPD anti-fade solution, Thermo Fisher Scientific). Confocal microscopy was performed on a Leica Confocal Microscope TCS SP-5 controlled by LAS-AF software using 10× and 63× (oil immersion) objectives (Leica Microsystems, Mannheim, Germany), with excitation/emission 499/520 nm for vimentin and α-SMA (Alexa488), 565/576 nm for PDGFRα (Alexa647), and 350/470 nm for DAPI.

Data from the above experiments are presented as mean±standard deviation and were analyzed by ordinary one-way analysis of variance (ANOVA) using GraphPad Prism 9.2.0 for comparison between different groups or between different days within the same initial cell seeding density group. p<0.05 was considered to be statistically significant. Each experiment was repeated at least three times for NIH 3T3 fibroblasts and twice for primary E16 mesenchymal cells.

The effect of the syringe volume/capacity for construction of Syringe-in-Syringe (SiS) devices on the diameter of hydrogel microstrands was also demonstrated. To investigate the feasibility of using the SiS device to fabricate implantable alginate hydrogel microstrands with a diameter around 200 μm for cell delivery and implantation studies, different syringe combinations of SiS devices were used. Specifically, variations in the capacity of the alginate syringe (1 mL, 3 mL, and 5 mL) and cross-linker syringe (1 mL, 3 mL, 5 mL, and 10 mL) while keeping the needle size constant (30G, inner diameter 159 μm). While the actual volume of alginate solution (1.5% w/v, kinematic viscosity measured to be 24.5+0.06 mm²/s at room temperature) loaded in each alginate syringe remained constant at 250 μL, we loaded each cross-linker syringe with 100 mM CaCl₂) in the actual volume around 70% of its full capacity. Analyzing the diameters of the resulting microstrands generated from these SiS devices with different combinations allowed us to determine the feasibility of fabricating alginate hydrogel microstrands with various combinations and to assess the reproducibility of this fabrication method, as shown in Table 1.

TABLE 1
Diameter of alginate hydrogel microstrands fabricated by the Syringe-in-Syringe devices (similar to device 10).

| | Alginate | Cross-linker | | | | | | |
|---|---|---|---|---|---|---|---|---|
| | syringe | syringe | Needle | | | | | |
| Device | capacity | capacity | gauge | Diameter of microstrands (μm)^{a,}*** | | | | |
| No. | (mL) | (mL) | (G) | Set 1 | Set 2 | Set 3 | Set 4 | Average |
| 1 | 1 | 1 | 30 | not feasible | | | | |
| 2 | 1 | 3 | 30 | 290.7 ± 13.7 | 271.0 ± 7.4 | 295.0 ± 4.4 | 310.4 ± 9.5 | 291.8 ± 16.2 |
| 3 | 1 | 5 | 30 | 253.5 ± 8.6 | 213.4 ± 7.4 | 208.4 ± 1.7 | 211.2 ± 4.7 | 221.6 ± 21.3 |
| 4 | 1 | 10 | 30 | 202.3 ± 3.1 | 189.0 ± 1.7 | 183.8 ± 5.2 | 187.6 ± 4.4 | 190.7 ± 8.1 |
| 5 | 3 | 3 | 30 | not feasible | | | | |
| 6 | 3 | 5 | 30 | not feasible | | | | |
| 7 | 3 | 10 | 30 | 309.0 ± 3.6 | 311.9 ± 1.5 | 299.7 ± 3.6 | 299.7 ± 7.8 | 303.8 ± 5.7 |

| | | | | |
|---|---|---|---|---|
| 8 | 5 | 5 | 30 | not feasible |
| 9 | 5 | 10 | 30 | not feasible |

^{a}Mean ± standard deviation (n = 3)
***p < 0.0001 between the average diameters of microstrands made by device No. 2, 3, 4, and 7.

Successful microstrand fabrication using SiS devices (such as device 10) occurs when the ratio of the alginate syringe volume/capacity to the cross-linker syringe volume/capacity was 1:3 or higher (i.e., 1 mL/3 mL, 1 mL/5 mL, 1 mL/10 mL, 3 mL/10 mL syringe combination). This ratio is more optimal for creating the necessary negative pressure to pull the alginate solution 16 from the alginate first syringe 12 through the connecting needle into the CaCl₂) cross-linker 18 solution in the cross-linker second syringe 14 to form the microstrands (Table 1). While keeping the needle size, volume of alginate used, and alginate first syringe 12 capacity constant, increasing the capacity of the cross-linker second syringe 14 led to a decrease in the diameter of microstrands. Additionally, increasing the capacity of the alginate syringe from 1 mL to 3 mL while keeping the cross-linker second syringe 14 capacity at 10 mL increased the diameter of microstrands. In particular, using 1 mL/3 mL, 1 mL/5 mL, 1 mL/10 mL, and 3 mL/10 mL SiS devices, one can repeatedly produce alginate hydrogel microstrands with a similar diameter for each device and with a standard deviation less than 10% of the average diameter, indicating the reproducibility of fabricating alginate hydrogel microstrands using these SiS devices.

Using the 1 mL/5 mL and 1 mL/10 mL SiS devices allowed us to fabricate microstrands with diameters around 200 μm, generally believed to be the limit for adequate oxygen mass transfer [76], showcasing the potential for fabricating hydrogel-based cell-delivery vehicles for implantation studies using a syringe-based handheld SiS device. Compared to 1 mL/10 mL SiS devices, 1 mL/5 mL devices generated less negative pressure for microstrand formation, making the fabrication process better controlled. Therefore, we chose 1 mL/5 mL SiS devices for the subsequent studies. The morphology of microstrands made by 1 mL/5 mL SiS devices was determined by SEM, in which the air-dried sample showed a smooth surface with a compact network-like structure on the surface and the lyophilized sample showed a rough surface with internal porous structure.

To determine the effectiveness of using the SiS device for cell delivery and implantation research, we encapsulated cells in alginate hydrogel microstrands with a known cell number, released the cells from the microstrands after a three-hour incubation, and determined the cell encapsulation and recovery efficiencies. Specifically, we utilized NIH 3T3 fibroblasts as a stromal cell model at varying initial cell seeding densities (1-8×10⁶ cells/mL alginate solution) and encapsulated cells in alginate hydrogel microstrands using the 1 mL/5 mL SiS device. Following a three-hour incubation period post-fabrication, these cell-laden microstrands were dissolved using 55 mM sodium citrate solution to release the cells. The total cell number and viable cell number recovered from each set of alginate hydrogel microstrands were measured using trypan blue exclusion assay. We calculated the encapsulation and recovery efficiency by dividing the total cell number after release from each set of microstrands by the initial cell number prior to encapsulation.

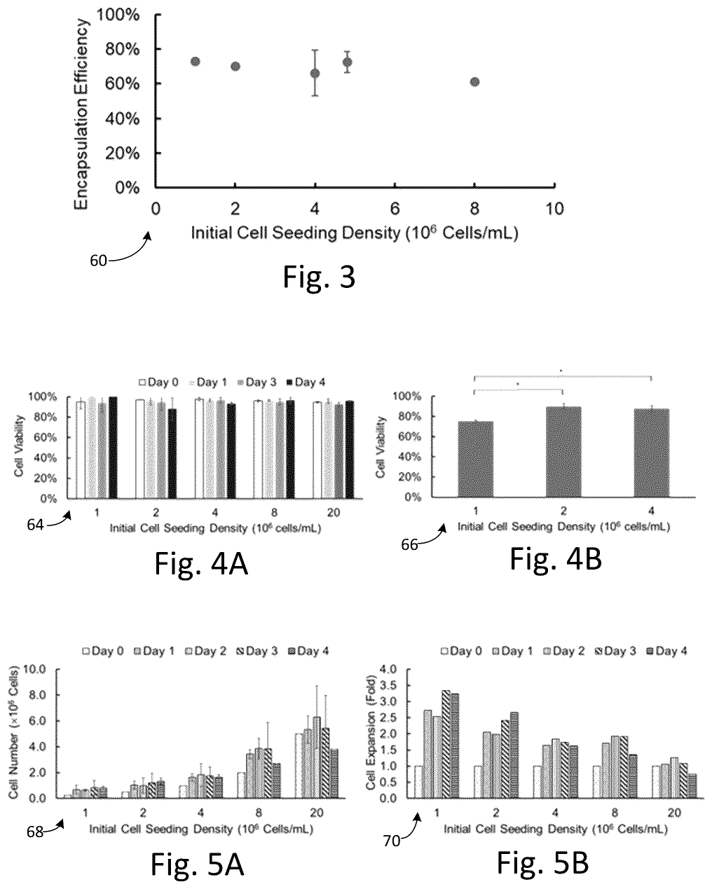

The encapsulation and recovery efficiency averaged 69+6% across different initial cell seeding densities, ranged from 1 to 8×10⁶ cells/mL alginate solution as shown in the graph 60 of FIG. 3. FIG. 3 is a graph illustrating the efficiency of cell encapsulation and recovery from alginate hydrogel microstrands containing NIH 3T3 fibroblasts at different initial cell seeding densities.

The cell viability of NIH 3T3 fibroblasts and primary E16 mesenchyme cells encapsulated in alginate hydrogel microstrands were assessed during 4- and 3-day growth periods, respectively. NIH 3T3 fibroblasts (1×10⁶ cells) were encapsulated in 250 μL alginate hydrogel microstrands and cultured for 0, 1, 3, or 4 days with culture medium changed daily. Cell viability of NIH 3T3 fibroblasts in microstrands remained ≥90% for four days. Next, the effect of initial cell seeding density on cell viability during high density cell culture in microstrands was further evaluated.

The results demonstrated that over 90% of NIH 3T3 fibroblasts remained viable when encapsulated in the alginate hydrogel microstrands at various initial cell seeding densities ranging from 1 to 20×10⁶ cells/mL alginate and cultured for 0, 1, 3, and 4 days, as shown in FIG. 4A. FIG. 4A is a graph 64 illustrating viability of cells cultured in alginate hydrogel microstrands with NIH 3T3 fibroblasts encapsulated in microstrands at various initial cell seeding densities. Graph 64 illustrates NIH 3T3 fibroblasts encapsulated in microstrands at various initial cell seeding densities (1-20×10⁶ cells/mL alginate), cultured for 0, 1, 3 and 4 days.

The viability of NIH 3T3 fibroblasts in alginate hydrogel microstrands at various initial seeding density were further confirmed by LIVE/DEAD assay, revealing that the majority of cells within the microstrands remained viable until day 4. Similarly, primary E16 mesenchyme cells at initial cell seeding densities ranging from 1-4×10⁶ cells/mL alginate were encapsulated in microstrands. While 1×10⁶ cells/mL alginate encapsulated microstrands exhibited 75% cell viability, 2×10⁶ cells/mL alginate and 4×10⁶ cells/mL alginate encapsulated microstrands exhibited over 90% cell viability on day 3 as shown in FIG. 4B. FIG. 4B is a graph 66 illustrating viability of cells cultured in alginate hydrogel microstrands with primary E16 mesenchyme cells encapsulated in microstrands at various cell seeding densities. Graph 66 illustrates primary E16 mesenchyme cells encapsulated in microstrands at various cell seeding densities (1-4×106 cells/mL alginate), cultured for 3 days*, p<0.05.

In addition, the LIVE/DEAD assay revealed that the majority of primary E16 mesenchyme cells within the microstrands at an initial seeding density of 4×10⁶ cells/mL alginate remained viable on day 4. From both optical images and fluorescence images of NIH 3T3 fibroblasts and E16 mesenchyme cells, we can tell that cells in the microstrands exhibited even distribution throughout.

Following a successful demonstration of high cell viability in alginate hydrogel microstrands for different cell types, the effect of initial cell seeding densities and culture duration on cell growth in microstrands using an alamarBlue assay can be shown. NIH 3T3 fibroblasts were encapsulated in alginate hydrogel microstrands at different initial cell seeding densities, ranging from 1-20×10⁶ cells/mL alginate solution and determined viable cell number on days 0, 1, 2, 3, and 4, respectively. The alamarBlue assay was used to measure cell number in microstrands on each day because alamarBlue allows for non-invasive, real-time monitoring of viable cell number. The effect of initial cell seeding densities (1, 2, 4, 8, and 20×10⁶ cells/mL alginate) on cell growth for NIH 3T3 fibroblasts in microstrands is shown in FIG. 5A. FIG. 5A is a graph 68 illustrating cell growth of NIH 3T3 fibroblasts in alginate hydrogel microstrands at different initial cell seeding densities through viable cell number.

At initial cell seeding densities of 1-8×10⁶ cells/mL alginate, the cell-laden microstrands showed an increase in cell number on day 1 and then reached a plateau for 4 days. For an initial cell seeding density of 2×10⁷ cells/mL alginate solution, the cell-laden microstrands exhibited maintenance of cell number for three days (FIG. 5A). For initial cell seeding densities of 8×10⁶ and 2×10⁷ cells/mL alginate, the cell number decreased on day 4 compared to day 3 although the difference was not significant, as shown in FIG. 5A.

The lower the initial cell seeding density, the greater the fold cell expansion is shown in FIG. 5B. FIG. 5B is a graph 70 illustrating cell growth of NIH 3T3 fibroblasts in alginate hydrogel microstrands at different initial cell seeding through cell expansion in fold.

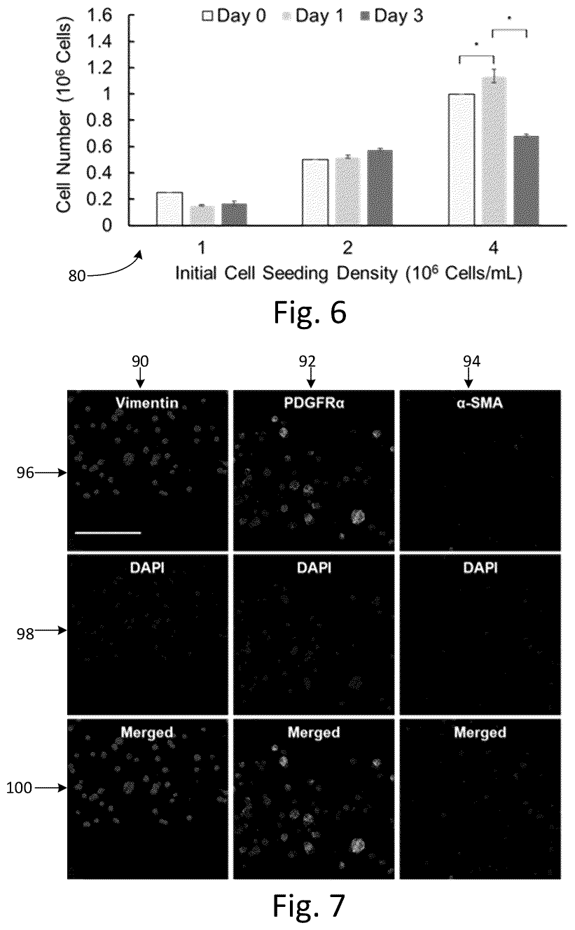

Cell growth in alginate hydrogel microstrands was assessed by alamarBlue assay using murine E16 salivary mesenchyme cells as a primary stromal model. Primary E16 mesenchyme cells were encapsulated in alginate hydrogel microstrands at different initial cell seeding densities (1, 2 and 4×10⁶ cells/mL alginate) using the 1 mL/5 mL SiS device and cultured for 3 days, as illustrated in FIG. 6. FIG. 6 is a graph 80 illustrating cell growth of primary E16 mesenchyme cells in alginate hydrogel microstrands. Graph 80 shows cell growth of primary E16 mesenchyme cells in 250 μL alginate hydrogel microstrands deter-mined by alamarBlue assay on days 0, 1, and 3. *, p<0.05. When the initial cell seeding density was 1×10⁶ cells/mL alginate solution, the cell number did not increase with culture duration, but when the initial cell seeding density was 2×10⁶ cells/mL alginate solution, the cell number increased with culture period. When the initial cell seeding density was 4×10⁶ cells/mL alginate solution, the cell number increased on day 1 but decreased on day 3. These data suggest that can encapsulate primary stromal cells at 2×10⁶ cells/mL alginate solution and culture these cell-laden microstrands up to 3 days or encapsulate cells at 4×10⁶ cells/mL alginate solution and culture for 1 day prior to cell implantation.

Maintenance of the endogenous E16 stromal mesenchymal cell markers vimentin and PDGFRα, and the myofibroblast fibrotic marker α-SMA in alginate hydrogel microstrands was also assessed. When 1×10⁶ primary E16 mesenchyme cells were grown in 250 μL alginate hydrogel microstrands (i.e., 4×10⁶ cells/mL alginate solution) for 4 days, they maintained expression of native stromal mesenchymal markers, vimentin and PDGFRα, with minimal expression of α-SMA, as shown in FIG. 7.

FIG. 7 is a series of confocal images of primary E16 mesenchyme cells cultured in alginate hydrogel microstrands. In FIG. 7, confocal images of primary E16 mesenchyme cells cultured in microstrands showing expression of mesenchymal markers, vimentin (column 90) and PDGFRα (column 92) while showing minimal expression of the fibrotic marker, α-SMA (column 94) on day 4 (row 96). Middle row 98 shows DAPI-stained cell nuclei in each group. Bottom 100 shows the merged images of rows 96 and 98. The images are at scale bar=100 μm. These results show that alginate hydrogel microstrands supported viable cell growth and retention of homeostatic mesenchymal phenotype, without a conversion to a myofibroblast-like phenotype.

Accordingly, successful MSC-based therapies may benefit from effective delivery and retention of cells at the target site, which necessitates the development of suitable delivery vehicles. The presentive invention strategically deals with the fabrication and evaluation of alginate hydrogel microstrands for cell survival, growth, and phenotype maintenance for their potential use as cell delivery vehicles for MSCs.

The handheld SiS device (device 10, FIG. 1) provides a simple and straightforward approach to fabricating implantable, cell-laden alginate hydrogels microstrands or microfibers. Current major methods to fabricate alginate hydrogel microfibers include microfluidics, extrusion, and 3D bioprinting. 3D bioprinting features precise control over the fabrication of intricate and complex structures by printing layer-by-layer, which cannot be attained by conventional fabrication strategies. However, bioinks in 3D printers have limitations including the requirement for pre-crosslinked bioink forms, the need for biocompatible formulations, the necessity for precise control over cell distribution and organization, and the optimization of bioinks for specific cellular applications. Along with the long fabrication time, the bioink operation temperatures and the inability to handle high cell densities could cause damage to cells during 3D printing. The clogging of needle tips in 3D printing is another significant problem faced in extrusion and extrusion-based bioprinting, particularly at high cell densities, which can cause cell over-accumulation both in the print head and in small features.

Microfluidics are also a common method for the fabrication of alginate hydrogel microfibers. However, significant time is required to learn the process and design a device to match/optimize the flow rates in each channel. In the present approach to using the SiS device (device 10), the uniformly mixed cell-alginate solution is loaded in the alginate first 12 syringe and 100 mM CaCl₂) solution is loaded in the cross-linker second syringe 14. By simply withdrawing the cross-linker second syringe 14, a negative pressure is created that pulls the cell-alginate solution from the alginate first 12 syringe through the connecting needle into the cross-linker second syringe 14. The negative pressure appears to prevent clogging of the needle tip.

Here, present device 10 is also able to handle high cell densities up to 20 million cells per mL alginate solution 16 with an encapsulation and recovery efficiency around 70%. The encapsulation efficiency could be potentially improved by utilizing zero-void syringes, which would be further optimized to minimize cell loss during encapsulation for cell delivery and implantation. The device 10 permits alginate-hydrogel microstrand fabrication in a couple minutes with high reproducibility. An ideal cell delivery vehicle provides a 3D environment for transplanted cells and enables them to maintain their viability and phenotypic characteristics, allowing them to restore the function of damaged or deceased tissues. Here, the alginate hydrogel microstrands are able to support cell viability and retention of endogenous cell phenotype markers. Altogether, the present device 10 generates consistent and controlled microstrands for various research applications, including cell delivery and implantation of cells in vivo for regenerative medicine approaches.

In another application of therapy, the development of xerostomia or dry mouth syndrome is often accompanied by hyposalivation and fibrosis, which can lead to permanent scarring and organ dysfunction. Despite extensive research, current pharmacological interventions have not been highly successful in treating xerostomia. Stem cell-based therapies, particularly using MSCs, have shown promise in regenerating salivary gland function due to their regenerative, anti-fibrotic, and anti-inflammatory properties. The usefulness of MSCs for salivary gland regeneration has been demonstrated by injection of MSCs into damaged salivary glands in humans or through the tail vein in mouse models. However, clinical application of MSCs requires high cell expansion in scaffold systems prior to transplantation, and in vivo inflammatory molecules or environment have adverse effects on immunogenicity, viability, and differentiation capacity of MSCs. To counteract MSC clearance by the recipient immune system, retention and homing of transplanted MSCs is necessary. Other bioengineered approaches have been tried for salivary gland regeneration, including controlled release of drugs such as pilocarpine and cevimeline, gene therapy, and fabrication of different biomaterials. These approaches are limited by adverse side effects, mutagenesis, and host tissue responses to the transferred biomaterials, respectively. The present invention can therefore be used in therapy and treatment of xerostomia.

Alginate hydrogels have been used for MSC delivery and implantation for different organ regeneration studies, such as chondrogenesis, stomach wall regeneration, bone, and nerve regeneration. Recently, alginate hydrogels have been used as drug delivery vehicles for regeneration of salivary glands. In particular, alginate hydrogel microstrands provide advantages of high cell density encapsulation, high cell viability, controlled cell alignment, enhanced cell-cell interactions, high surface-to-volume ratio, ease in handling and manipulation. Most studies using alginate hydrogel microfibers for in vivo implantation incorporated ECM proteins or growth factors to support cell growth. The present invention provides the ability to support cell viability above 90% using alginate hydrogels alone, providing a simple alginate hydrogel system that could be used in implantation studies, treatments, and therapies.

The corresponding structures, materials, acts, and equivalents of all means or step plus function elements in the claims below, if any, are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of one or more aspects of the invention and the practical application, and to enable others of ordinary skill in the art to understand one or more aspects of the invention for various embodiments with various modifications as are suited to the particular use contemplated.

What is claimed is:

1. A device for fabricating alginate hydrogel microstrands, comprising:
a first reservoir including a cell-alginate solution therein, the first reservoir configured to selectively intake and expel the cell-alginate solution therefrom;
a second reservoir in fluid connection with the first reservoir, the second reservoir containing a cross-linker solution and configured to selectively intake the cell-alginate solution from the first reservoir and expel a combined cell-alginate solution and cross-linker solution therefrom; and
wherein the second reservoir configured to selectively expel the combined cell-alginate solution and cross-linker solution as cell-laden hydrogel microstrands.

2. The device of claim 1, wherein the second reservoir selectively detaches at a nozzle from the first reservoir, the nozzle configured to expel the cell-laden hydrogel microstrands adjacent to one or more mammalian cells.

3. The device of claim 1, wherein the cell-alginate solution and cross-linker solution are combined at a predetermined duration to form cell-laden alginate hydrogel microstrands with long, thin fiber-like structures.

4. The device of claim 3, wherein the predetermined duration is in a range of 5 seconds to 50 seconds.

5. The device of claim 1, wherein first reservoir and second reservoir are each a syringe having a chamber with manually operated plungers therein, and each syringe including a nozzle thereof, and wherein a first nozzle of the first syringe and a second nozzle of the second syringe are selectively attached to thereby create a fluid coupling between a first chamber and a second chamber.

6. The device of claim 1, wherein the cell-alginate solution includes one of: NIH 3T3 cells, epithelial cells, endothelial cells, neural cells, or stem cells.

7. The device of claim 1, wherein the cross-linker solution includes one of Ca²⁺, CaCl₂, Ba²⁺, Sr²⁺.

8. The device of claim 1, further including a filter having micropatterned pores connecting the first reservoir and second reservoir.

9. A process for fabricating alginate hydrogel microstrands, comprising:
placing a cell-alginate solution in a first reservoir, the first reservoir configured to selectively intake and expel the cell-alginate solution therefrom;
placing a cross-linker solution in a second reservoir in fluid connection with the first reservoir, the second reservoir configured to selectively intake the cell-alginate solution from the first reservoir and expel the combined cell-alginate solution and cross-linker solution therefrom;
intaking the cell-alginate solution from the first reservoir into the second reservoir;
allowing the cell-alginate solution and the cross-linker solution to mix for a predetermined duration; and
after the predetermined duration, expelling a combined cell-alginate solution and cross-linker solution as cell-laden hydrogel microstrands from the second reservoir.

10. The process of claim 9, wherein the second reservoir selectively detaches at a nozzle from the first reservoir, and further comprising:
detaching the nozzle from the first reservoir; and
expelling the cell-laden hydrogel microstrands adjacent to one or more mammalian cells.

11. The process of claim 9, wherein the predetermined duration is in a range of 5 seconds to 50 seconds.

12. The process of claim 9, wherein first chamber and second chamber are each a syringe having manually operated plungers therein, and each syringe including a nozzle thereof, and further comprising selectively attaching the first nozzle of the first syringe and second nozzle of the second syringe to thereby create a fluid coupling between the first chamber and second chamber.

13. The process of claim 9, wherein the cell-alginate solution includes NIH 3T3 cells.

14. The process of claim 9, further including expelling the combined cell-alginate solution and cross-linker solution through a filter having micropatterned pores therein.

15. The process of claim 10, further comprising incubating the microstrands predetermined duration prior to expelling the cell-laden hydrogel microstrands adjacent to one or more mammalian cells.

16. The process of claim 10, wherein the cell-laden hydrogel microstrands are expelled into at least one cell culture well plate.

17. The process of claim 12, wherein the intaking cell-alginate solution and and expelling of combined cell-alginate solution and cross-linker solution are achieved by action on the manually operated plungers.

18. The process of claim 14, wherein the filtering is performed through a separate filter from the first reservoir and second reservoir.

19. The process of claim 14, wherein a filter is located at the nozzle of the second reservoir, and the filtering is performed by expelling the combined cell-alginate solution and cross-linker solution from the second reservoir.

20. A solution containing cell-laden hydrogel microstrands with long and thin fiber-like structures, produced by the process of claim 9.
